(19) United States
(12) Patent Application Publication
(10) Pub. No.: US 2024/0206907 A1
(43) Pub. Date: Jun. 27, 2024

(54) System and Method for Device Tracking in Magnetic Resonance Imaging Guided Inerventions

(71) Applicant: The Regents of the University of California, Oakland, CA (US)

(72) Inventors: Holden H. Wu, Los Angeles, CA (US); Xinzhou Li, Los Angeles, CA (US); David S. Lu, Manhattan Beach, CA (US)

(21) Appl. No.: 18/558,118

(22) PCT Filed: May 2, 2022
(86) PCT No.: PCT/US2022/027339

§ 371 Date: Oct. 30, 2023

Related U.S. Application Data
(60) Provisional application No. 63/182,754, filed on Apr. 30, 2021.

(51) Int. Cl. A61B 17/34; A61B 34/20; A61B 90/00

(52) U.S. Cl. CPC A61B 17/3403; A61B 34/20; A61B 90/37; A61B 2034/2065; A61B 2034/2074; A61B 2090/374

(57) ABSTRACT

In accordance with an embodiment, a system for device localization and tracking for magnetic resonance (MR) image-guided interventional procedures includes an input configured to receive a set of MR images of a region of interest of a subject acquired using a magnetic resonance imaging (MRI) system and a physical device localization system. The region of interest includes a device. The physical device localization system includes a first neural network coupled to the input and configured to localize a feature of the device on the set of MR images and a second neural network coupled to the first neural network and configured to generate an estimate of a physical position of the device based on the localized device feature. The system further includes a display coupled to the second neural network and configured to display the estimate of the physical position of the device.

### CROSS-REFERENCE TO RELATED APPLICATIONS

This application is based on, claims priority to, and incorporates herein by reference in its entirety U.S. Ser. No. 63/182,754 filed Apr. 30, 2021 and entitled “Methods for Device Tracking in Magnetic Resonance Imaging Guided Interventions.”

### BACKGROUND

The success of minimally invasive image-guided percutaneous procedures (or interventions), such as targeted biopsy and focal ablation, depends on intra-procedural imaging to visualize tissue and devices (e.g., needles) simultaneously for guidance and confirmation. Image-guided percutaneous interventional procedures can play a key role in the diagnosis and treatment of various diseases, for example cancer. Under image guidance, physicians manipulate devices (e.g., needles) to access targets in, for example, suspected lesions for different purposes. Magnetic resonance imaging (MRI) has multiple advantages for intra-procedural imaging, including high soft tissue contrast, flexible plane orientations, and no ionizing radiation. In addition, recent developments of robotic needle control based on real-time MRI have potential to achieve dynamic needle placement with high accuracy. Automatic, accurate, and rapid needle localization will be required for needle adjustment under both intra-procedural and real-time MRI guidance. Needle localization and tracking are essential for the safety, accuracy, and efficiency of MRI guided percutaneous interventions.

Intra-procedural or real-time MRI scans may be used to visualize both the needle feature and the tissue target to assist needle position adjustments during interventions. Passive needle visualization relies on MR image features (e.g., signal void) caused by the needle-induced susceptibility effects to estimate the needle position. Compared with active needle visualization in MRI, which requires additional hardware, passive needle visualization methods only require image processing. However, automatic needle localization based on the passive needle feature is challenged by variations of the needle susceptibility-induced signal void feature due to different situations and due to contrast between the needle feature and surrounding tissue, both of which can lead to inaccurate interpretation of the needle position. This may result in several iterations of adjustments of the needle position, or even errors in the needle placement relative to the tissue target.

The susceptibility difference between the needle and surrounding tissue can cause magnetic field perturbation and MR signal dephasing. With MR-compatible needle materials, such as titanium alloys, the needle susceptibility and geometry usually lead to a long tubular signal void feature on MR images. This needle signal void feature can have an irregular shape at the tip and the axis can be shifted from the physical needle axis. Therefore, even if the image plane is perfectly aligned with the needle, there may be discrepancies between the needle feature and physical needle. The discrepancies between the passive needle feature on MRI and the underlying physical needle position may induce errors in estimating the true needle position (needle localization) during procedures. Previous studies have reported that this discrepancy can reach 5-10 mm and can depend on the MRI sequence type and parameters, the needle material, and the needle's orientation relative to the B₀ field. Therefore, only localizing the needle feature to monitor the physical needle position during the procedure may cause substantial errors in needle targeting.

One previous approach developed to try to overcome this limitation is to reduce or correct the distortion of the signal void feature versus the physical object with multispectral MR imaging. For example, slice-encoding metal artifact correction (SEMAC) can minimize the average needle tip error (−0.4 mm) with improved time efficiency using compressed sensing (CS) reconstruction. However, the combined acquisition and reconstruction time of CS-SEMAC (˜30 sec) is not appropriate for immediate updates of the needle position for feedback during procedures. Another approach is to reconstruct the precise physical object shape by forming an inverse problem based on a set of acquisition MRI signals. The forward modeling of the needle susceptibility-induced signal void has been reported for difference sequence parameters and needle geometry. However, due to the ill-posed nature of the inversion problem, multi-orientation sampling and iterative computation, similar to strategies for quantitative susceptibility mapping, may be required, which are not practical for time-sensitive interventional procedures.

Supervised deep learning using convolutional neural networks (CNNs) is a potential approach to rapidly and accurately calculate solutions to ill-posed inversion problems involving magnetic susceptibility. For example, DeepQSM and QSMNet both use pixel-level semantic models based on U-Net to solve ill-posed field-to-source inversion problems and reconstruct quantitative tissue susceptibility maps from single-orientation MRI phase data with rapid inference time. These previous pixel-based CNN models aimed to solve for the tissue susceptibility map over the entire field of view (FOV) based on the phase map, but this may not be suitable for the physical needle localization problem, which requires local information about the device. In addition, pixel-level semantic methods could be sensitive to false detection of small objects (e.g., a needle segment in a full FOV image). Accordingly, these methods can require additional postprocessing to remove false positive needle feature detection results.

It would be desirable to provide a system and method for device localization and tracking that can accurately estimate both the device (e.g., a needle) feature and the physical device (e.g., a needle) position and that overcomes the challenges for device localization and tracking using MR images for interventional procedures.

### SUMMARY

In accordance with an embodiment, a system for device localization and tracking for magnetic resonance imaging (MRI)-guided interventional procedures includes an input configured to receive a set of MR images of a region of interest of a subject acquired using a magnetic resonance imaging (MRI) system and a physical device localization system. The region of interest may include a device. The physical device localization system includes a first neural network coupled to the input and configured to detect and localize a feature of the device on the set of MR images and a second neural network coupled to the first neural network and configured to generate an estimate of a physical position of the device based on the localized device feature. The system further includes a display coupled to the second neural network and configured to display the estimate of the physical position of the device.

In accordance with another embodiment, a method for device localization and tracking for magnetic resonance imaging (MRI) image-guided interventional procedures includes acquiring a set of MR images of a region of interest of a subject. The region of interest may include a device. The method further includes detecting and localizing a feature of the device on the set of MR images using a first neural network, generating an estimate of a physical position of the device based on the localized device feature using a second neural network coupled to the first neural network and displaying, using a display, the estimate of the physical position of the device.

### BRIEF DESCRIPTION OF THE DRAWINGS

The present invention will hereafter be described with reference to the accompanying drawings, wherein like reference numerals denote like elements.

FIG. 1 is a schematic diagram of an example MRI system in accordance with an embodiment;

FIG. 2 is a block diagram of a system for device localization and tracking on magnetic resonance (MR) images in accordance with an embodiment;

FIGS. 3A-3C illustrate a method for device localization and tracking on MR images in accordance with an embodiment;

FIG. 4 is a block diagram of a device feature mask region-based convolutional neural network (Mask R-CNN) of the system for device localization and tracking of FIG. 2 in accordance with an embodiment;

FIG. 5 is a block diagram of a physical device Mask R-CNN of the system for device localization and tracking of FIG. 2 in accordance with an embodiment;

FIG. 6A shows an example physical needle detection and segmentation output for a physical needle Mask R-CNN in accordance with an embodiment;

FIG. 6B shows an example physical needle localization output for a system for needle localization based on the physical needle detection and segmentation output of FIG. 6A in accordance with an embodiment;

FIG. 7 is a block diagram of a system for automatic MRI scan plane alignment in accordance with an embodiment;

FIG. 8 illustrates a method for automatic MRI scan plane alignment in accordance with an embodiment; and

FIG. 9 is a block diagram of an example computer system in accordance with an embodiment.

### DETAILED DESCRIPTION

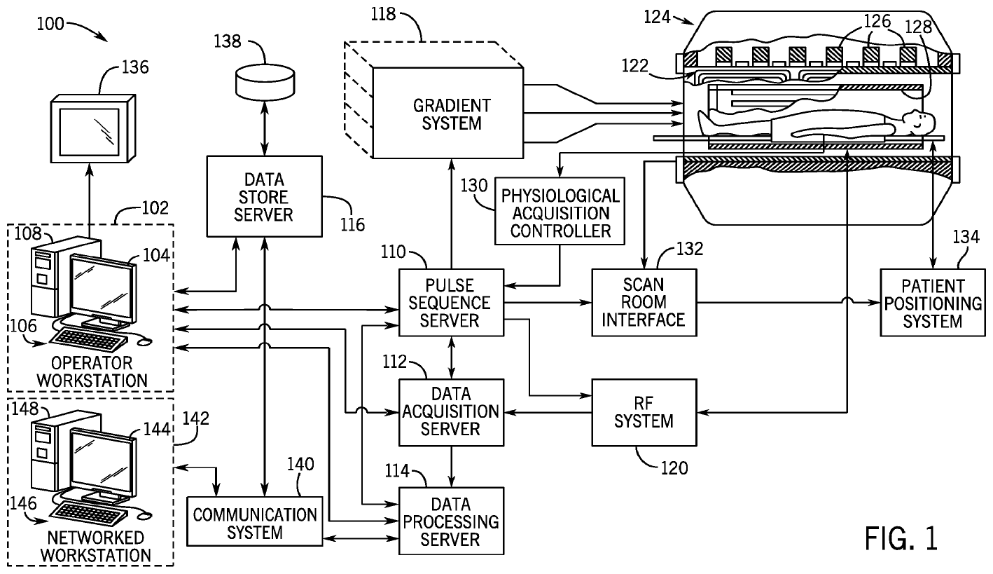

FIG. 1 shows an example of an MRI system 100 that may be used to perform the methods described herein. MRI system 100 includes an operator workstation 102, which may include a display 104, one or more input devices 106 (e.g., a keyboard, a mouse), and a processor 108. The processor 108 may include a commercially available programmable machine running a commercially available operating system. The operator workstation 102 provides an operator interface that facilitates entering scan parameters into the MRI system 100. The operator workstation 102 may be coupled to different servers, including, for example, a pulse sequence server 110, a data acquisition server 112, a data processing server 114, and a data store server 116. The operator workstation 102 and the servers 110, 112, 114, and 116 may be connected via a communication system 140, which may include wired or wireless network connections.

The pulse sequence server 110 functions in response to instructions provided by the operator workstation 102 to operate a gradient system 118 and a radiofrequency (“RF”) system 120. Gradient waveforms for performing a prescribed scan are produced and applied to the gradient system 118, which then excites gradient coils in an assembly 122 to produce the magnetic field gradients Gₓ, G_{y}, and G_{Z} that are used for spatially encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil to perform the prescribed magnetic resonance pulse sequence. Responsive magnetic resonance signals detected by the RF coil 128, or a separate local coil, are received by the RF system 120. The responsive magnetic resonance signals may be amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the prescribed scan and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. An RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²} Eqn. 1;

and the phase of the received magnetic resonance signal may also be determined according to the following relationship:

$$φ = {tan}^{- 1} ( \frac{Q}{I} ) . \qquad Eqn 2$$

The pulse sequence server 110 may receive patient data from a physiological acquisition controller 130. By way of example, the physiological acquisition controller 130 may receive signals from a number of different sensors connected to the patient, including electrocardiograph (“ECG”) signals from electrodes, or respiratory signals from a respiratory bellows or other respiratory monitoring devices. These signals may be used by the pulse sequence server 110 to synchronize, or “gate,” the performance of the scan with the subject's heart beat or respiration.

The pulse sequence server 110 may also connect to a scan room interface circuit 132 that receives signals from various sensors associated with the condition of the patient and the magnet system. Through the scan room interface circuit 132, a patient positioning system 134 can receive commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, so that data is not lost by data overrun. In some scans, the data acquisition server 112 passes the acquired magnetic resonance data to the data processor server 114. In scans that require information derived from acquired magnetic resonance data to control the further performance of the scan, the data acquisition server 112 may be programmed to produce such information and convey it to the pulse sequence server 110. For example, during pre-scans, magnetic resonance data may be acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also process magnetic resonance signals used to detect the arrival of a contrast agent in a magnetic resonance angiography (“MRA”) scan. For example, the data acquisition server 112 may acquire magnetic resonance data and processes it in real-time to produce information that is used to control the scan.

The data processing server 114 receives magnetic resonance data from the data acquisition server 112 and processes the magnetic resonance data in accordance with instructions provided by the operator workstation 102. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 114 are conveyed back to the operator workstation 102 for storage. Real-time images may be stored in a data base memory cache, from which they may be output to operator display 104 or a display 136. Batch mode images or selected real time images may be stored in a host database on disc storage 138. When such images have been reconstructed and transferred to storage, the data processing server 114 may notify the data store server 116 on the operator workstation 102. The operator workstation 102 may be used by an operator to archive the images, produce films, or send the images via a network to other facilities.

The MRI system 100 may also include one or more networked workstations 142. For example, a networked workstation 142 may include a display 144, one or more input devices 146 (e.g., a keyboard, a mouse), and a processor 148. The networked workstation 142 may be located within the same facility as the operator workstation 102, or in a different facility, such as a different healthcare institution or clinic.

The networked workstation 142 may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may be exchanged between the data processing server 114 or the data store server 116 and the networked workstations 142, such that the data or images may be remotely processed by a networked workstation 142.

The present disclosure describes systems and methods for device localization and tracking on magnetic resonance (MR) images that utilize a deep learning-based framework to automatically and rapidly localize a physical device position based on the device features (i.e., passive device features) on MR images. In some embodiments, the deep learning framework is a two-stage deep learning-based framework for device localization that includes a device feature first neural network and a physical device neural network. In some embodiments, the two-stage deep learning based framework for device localization can be configured to localize a physical device position at the instance-level using mask region-based convolutional neural networks (Mask R-CNNs). The two-stage device localization system can include a first stage implemented as a device feature neural network (e.g., a CNN such as a Mask R-CNN) that is configured to detect and segment the device feature on an input MR image or image(s) and a second stage implemented as physical device neural network (e.g., a CNN such as a Mask R-CNN) that is configured to directly estimate the physical device position based on the device feature detected and segmented by the device feature neural network. Accordingly, the device feature neural network and the physical device neural network are combined to form an automatic framework to localize the physical device position on an MR image. In some embodiments, an image patch containing the device feature may be generated using the MR image input into the device feature neural network (e.g., a Mask R-CNN) and the device feature detection and segmentation results of the device feature neural network. The image patch may be used as the input to the physical device neural network (e.g., a Mask R-CNN). In some embodiments, the physical device neural network (e.g., a Mask R-CNN) may be trained and configured to receive and analyze a single-slice input, for example, the input image patch is a single slice. Accordingly, the single-slice physical device neural network can be configured to localize an in-plane 2D physical device tip and axis. In some embodiments, the physical device neural network may be trained and configured to receive and process three adjacent and parallel slices, for example, the input image patch consists of three patch(es) for three adjacent and parallel slices. Accordingly, the 3-slice physical device neural network can be configured to localize a 3D physical device tip and axis position (in-plane and through-plane).

In some embodiments, the physical device neural network, e.g., a Mask R-CNN, (single-slice or 3-slice) may be trained for physical device localization using simulated training data generated using physics-based simulations. For example, physics-based simulations may be used to generate single-slice or 3-slice images with device features from a range of underlying device positions and MRI parameters to form the training datasets. In some embodiments, the training of a single-slice physical device neural network (e.g., a Mask R-CNN) may be fine-tuned using a further training dataset generated using MR images combined with simulated needle features.

In some embodiments, the system and method for device localization and tracking (including the two-stage device localization system) can be used for MRI guided-percutaneous interventions (e.g., targeted biopsy or focal ablation). In some embodiments, the device localization and tracking system may be used to localize and track devices used in an MRI-guided intervention such as, for example, needles, ablation probes, monitoring probes, catheters, guidewires, etc. In some embodiments, the disclosed system and method for device localization and tracking may be used in real-time during one or more of the stages of an interventional procedure, for example, the system and method may be combined with planning images, pre-procedure images or intra-procedural images to directly facilitate the different stages of the procedure. Advantageously, during an interventional procedure, the disclosed system and method can, for example, improve the navigation of a device towards a target or improve the workflow for adjusting the device position. In some embodiments, the disclosed system and method may be applied to exiting images collected from an interventional procedure to perform post-procedural analysis of, for example, the device trajectory. For example, in some embodiments, the disclosed system and method may be used as a post-processing tool for device path analysis using procedural images to provide information about device trajectory and placement accuracy to improve procedural planning. In some embodiments, the disclosed system and method may be used to provide feedback information for manual manipulation of the device, robotic-assisted device control (e.g., for adjustment of device trajectory) or MRI scan parameter adjustment. In some embodiments, the system and method for device localization and tracking may be used for automatic scan plane alignment during an interventional procedure to ensure the scan plane aligns with the device feature or the physical device position.

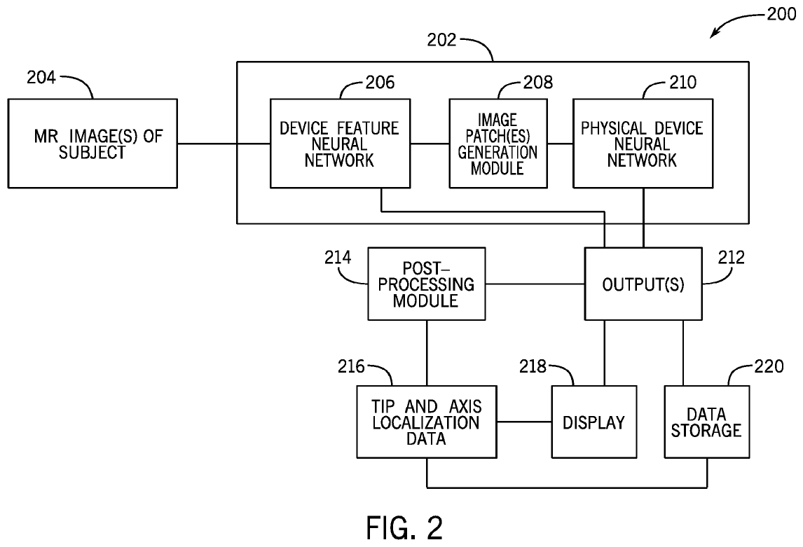

The following description of FIGS. 2-8 will discuss the systems and methods for device localization and tracking in terms of a needle device, however, it should be understood that, in some embodiments, the device localization and tracking system may be used to localize and track other devices used in an MRI-guided intervention such as, for example, ablation probes, monitoring probes, catheters, guidewires, etc. FIG. 2 is a block diagram of a system for device localization and tracking in accordance with an embodiment. The system 200 can include a two-stage physical device localization system 202, an input 204 of one or more magnetic resonance (MR) images of a subject, (e.g., a region of interest of a subject), output(s) 212 of the two-stage device localization system 202, post-processing module 214, an output 216 of the post-processing module 214 for tip and axis localization data, a display 218 and data storage 220. The input MR image(s) 204 of the subject may be acquired using an MRI system such as, for example, MRI system 100 shown in FIG. 1. In some embodiments, the input MR image(s) 204 may be grayscale 2D MR image(s) that are stacked into three color channels. In some embodiments, the input MR image(s) may be a stack of images encompassing the device, for example a needle, axis. Advantageously, in some embodiments, the two-stage device localization system 202 only requires an MR image (or images) as an input. In some embodiments, the MR image(s) 204 of the subject may be acquired in real-time from a subject (e.g., in vivo intra-procedural images) using an MRI system (e.g., MRI system 100 shown in FIG. 1), for example, for embodiments where the system 200 is utilized for device localization and tracking for MRI-guided interventions. For example, an MR image(s) 204 may be acquired from a subject using a pulse sequence performed on the MRI system and configured for interventional MRI. The interventional MRI sequence (and imaging parameters) used to acquire the input MR image(s) may be a known MRI interventional pulse sequence. In some embodiments, the input MR image(s) may be acquired using interactive real-time sequences configured to support a range of MRI-guided interventions. The acquired MR image(s) 204 may be provided to the two-stage physical device localization system 202 in real-time from an MRI system (e.g., from data processing server 114 or disc storage 138 of MRI system 100 shown in FIG. 1), for example, the MRI system 100 may acquire MR data and reconstruct an MR image or images (e.g., using data processing server 114) using known reconstruction methods. In some embodiments, the MR image(s) 204 of the subject may be retrieved from data storage (or memory) of system 200, data storage of an imaging system (e.g., data storage of MRI system 100 shown in FIG. 1), or data storage of other computer systems (e.g., storage device 816 of computer system 100 shown in FIG. 10). For example, for embodiments where the system 200 is utilized for post-procedural analysis of device trajectory, the input MR image(s) 204 may be MR images acquired during an interventional procedure (i.e., procedural images) and stored in data storage.

The input MR image(s) 204 of the subject may be provided as an input to the two-stage physical device localization system 202. In some embodiments, the two-stage physical device localization system 202 may be configured to generate a physical device localization (e.g., estimating the physical device position) based on a device feature (e.g., a needle signal void feature). In some embodiments, the physical device localization system 202 and system 200 are configured to advantageously provide an automatic physical device localization (e.g., on interventional MR images) for MRI-guided percutaneous interventions. In some embodiments, the two-stage physical device localization system 202 is implemented using convolutional neural networks such as, for example, Mask R-CNNs. Overall, in some embodiments, the disclosed two-stage physical device localization system 202 can, for example, be configured to overcome the discrepancy between the device feature and the physical device during interventional MRI procedures. In addition, in some embodiments, the physical device localization system 202 can used to automatically track a device (e.g., a needle) tip location and axis orientation. In some embodiments, the system 200 including physical device localization system 202 enable real time processing (e.g., ˜200 ms per image) that is, for example, acceptable for MRI-guided interventions.

As shown in FIG. 2, the two-stage device localization system 202 can include a first stage implemented using a device feature neural network (e.g., a CNN such as a Mask R-CNN) 206 and a second stage implemented as a physical device neural network) e.g., a CNN such as a Mask R-CNN) 210. As mentioned above the discrepancy between the device feature on MR images and the underlying physical device position may cause device localization or targeting errors, for example, for a needle on the order of 5-10 mm, which can be problematic, for example, for clinically relevant tumors with diameters of 5-10 mm. Advantageously, the disclosed technique includes as a second stage, a physics-driven physical device neural network (e.g., a CNN such as a Mask R-CNN) 210 to accurately localize the physical device (e.g., a needle) position based on the device feature extracted from the device feature neural network 206 of the first stage.

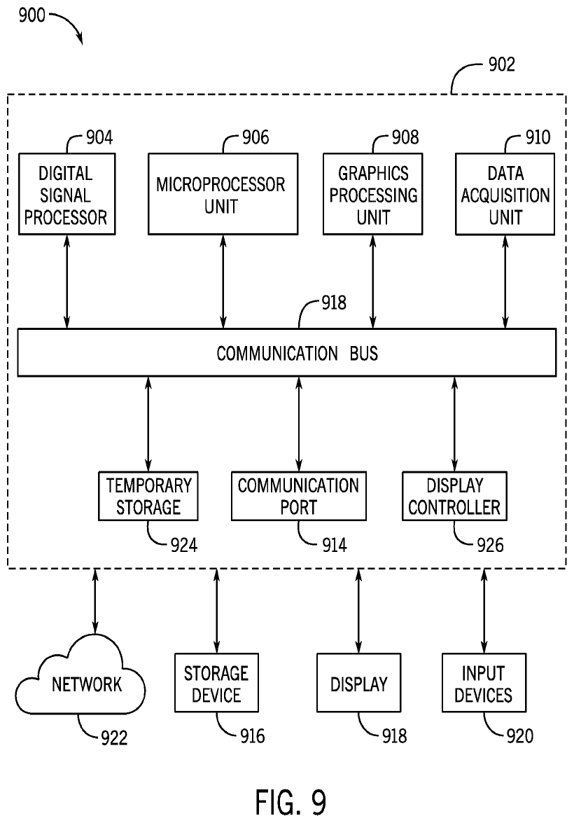

The input MR image(s) 204 may be provided to the device feature neural network 206 of the first stage. The device feature neural network 206 may be configured to detect and segment the device feature (e.g., the needle signal void feature) on the input MR image(s) 204. Advantageously, the device feature neural network 206 may detect and segment the device feature on the input MR image(s) at the instance-level (e.g., by implementing the device feature neural network as a Mask R-CNN). As mentioned above, in some embodiments, the input MR image(s) 204 are intra-procedural or real-time MR images. In some embodiments, the detection and segmentation process performed by the device feature neural network 206 can generate a plurality of outputs including a class score, a bounding box, and a predicted mask. For example, the bounding box may indicate the region of interest (ROI) corresponding to the device feature detection and the predicted mask (within the bounding box) may correspond to the device feature segmentation. In some embodiments, the plurality of outputs of the device feature neural network 206 may be stored in data storage, for example, data storage 220 (e.g., data storage of MRI system 100 shown in FIG. 1 or device storage 918 of computer system 900 shown in FIG. 9).

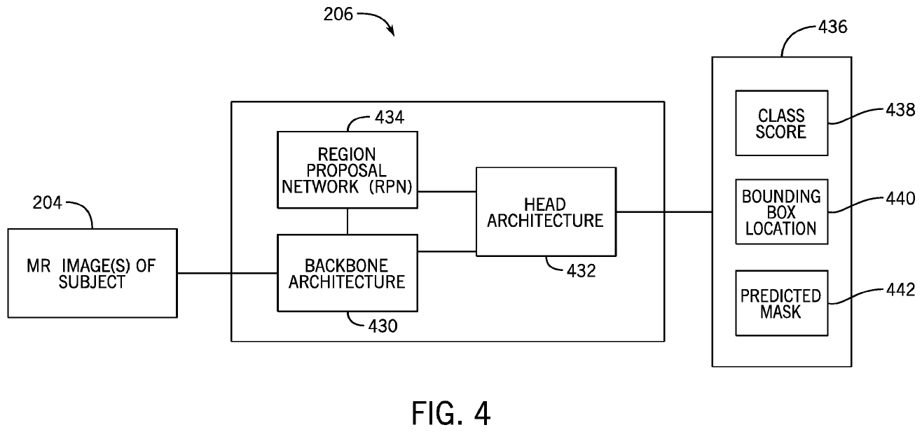

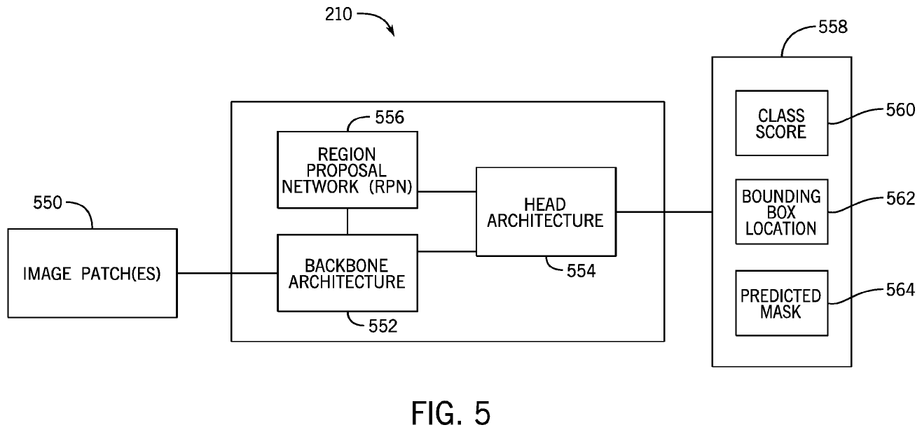

The detection and segmentation outputs (e.g., the class score, bounding box and predicted mask) generated by the device feature neural network 206 may be provided to an image patch generation module 208. In some embodiments, based on the results of the device feature neural network 206, the image patch generation module 208 may be configured to automatically crop the MR image(s) 204 to a patch centered on the device feature (e.g., as identified by the detection and segmentation outputs of the device feature neural network 206). In some embodiments, the cropped image patch is a single-slice image patch. In some embodiments, the image patch includes three adjacent and parallel image patches. In some embodiments, the cropped image patch can advantageously help to avoid false detection results and to maintain an assumption of a rigid device segment for the input to the physical device neural network models (for example, as discussed further below with respect to the example Mask R-CNN models illustrated in FIGS. 4 and 5). The cropped image patch (e.g., a single-slice image patch or three adjacent and parallel slices) may be provided as an input to the physical device neural network 210 of the second stage. In some embodiments, the physical device neural network 210 may be configured as a single-slice physical device Mask R-CNN model that takes a single-slice image patch as input. In some embodiments, the physical device neural network 210 may be configured as a 3-slice physical device Mask R-CNN model that takes the three adjacent and parallel image patches as input.

The physical device neural network 210 may be configured to generate a physical device localization including detecting the physical device (e.g., a needle) tip position and axis orientation. In some embodiments, the physical device neural network 210 can be trained as a single-slice physical device Mask R-CNN model which can take an image patch (e.g., a needle feature patch) from a single slice as input to localize the in-plane two-dimensional (2D) physical device (e.g., a needle) tip and axis. In some embodiments, input image patch may be a 2D gray-scale image patch and may be stacked into 3 color-channels to match the dimensions of the device feature Mask R-CNN 206. As discussed further below with respect to FIG. 4, the single-slice physical device Mask R-CNN model may include the assumption that the imaging plane is already closely aligned with the physical device. In some embodiments, the physical device Mask R-CNN 210 can be trained as a 3-slice physical device Mask R-CNN model which can be used to localize a three-dimensional (3D) physical device (e.g., a needle) position (in-plane and through plane) using a 3-slice image patch input. As discussed further below with respect to FIG. 5, the imaging plane orientation may be misaligned with the physical device axis in the input image patch for the 3-slice physical device Mask R-CNN model.

The physical device localization system 202 (e.g., from both the device feature neural network 206 and the physical device neural network 210) can generate a plurality of output(s) 212 including, for example, class score(s), bounding box(es), and a predicted masks. For example, as mentioned above, for the needle feature neural network 206 the bounding box may indicate the region of interest (ROI) corresponding to the device feature detection and the predicted mask (within the bounding box) may correspond to the device feature segmentation. For the physical device neural network 210, in an example, the bounding box may indicate the region of interest (ROI) corresponding to the physical device detection and the predicted mask (within the housing box) may correspond to the physical device segmentation. The output(s) may be displayed on a display 218 (e.g., displays 104, 136, 144 of the MRI system 100 shown in FIG. 1 or display 918 of the computer system 900 shown in FIG. 9). The outputs 212 may also be stored in data storage, for example, data storage 220 (e.g., data storage of MRI system 100 shown in FIG. 1 or device storage 918 of computer system 900 shown in FIG. 9).

Post-processing module 214 may be coupled to the physical device localization system 202 and may be configured to perform further processing on the outputs 212 (e.g., the segmentation mask) of the two-stage device localization system 202. In some embodiments, the post-processing module 214 may be configured to automatically determine or extract device feature tip and axis localization data 216 including, for example, an estimate of the device feature position or location of the device, based on the outputs (e.g., bounding box, segmentation mask) from the device feature neural network 206. In some embodiments, the post-processing module 214 may be configured to perform an orthogonal distance regression to localize the device feature tip and axis orientation. In an example where the device is a needle, the MRI needle signal void feature caused by susceptibility may be nearly symmetric, thus, the needle feature axis should be along the centerline of the segmentation mask (i.e., predicted mask of output(s) 212) generated from device feature neural network 206. For images with a positive detection of the needle feature, the location of the centerline can be estimated with the orthogonal distance regression using the entire needle feature segmentation mask. The signal drop along the detected axis may be used to identify the needle feature tip. The device feature tip and axis localization data 216 may be stored in data storage, for example, data storage 220. The device feature tip and axis localization data 216 may also be displayed on the display 218. Advantageously, the device feature position (e.g., tip position and axis orientation) estimated by system 200 can provide accurate feedback in real time during percutaneous interventions to guide or assist the physician and enable, for example, accurate manual device control or automatic remote control of device manipulation by robotic systems in the MRI system. In some embodiments, the device feature position data can also be used to update the MRI scan parameters of an MRI system (e.g., MRI system 100 shown in FIG. 1) to track and improve visualization of the device feature throughout different steps of the procedure

In some embodiments, the post-processing module 214 may be configured to automatically determine or extract physical device tip and axis localization data 216 including, for example, an estimate of the physical position or location of the device, based on the outputs (e.g., bounding box, segmentation mask) from the physical device neural network 210. In some embodiments, the post-processing module 214 may be configured to perform an orthogonal distance regression to localize the physical device tip and axis orientation. In an example where the device is a needle, the MRI needle signal void feature caused by susceptibility may be nearly symmetric, thus, the needle feature axis should be along the centerline of the segmentation mask (i.e., predicted mask of output(s) 212) generated from physical device neural network 210. For images with a positive detection of the physical needle, the location of the centerline can be estimated with the orthogonal distance regression using the entire physical needle segmentation mask. The signal drop along the detected axis may be used to identify the physical needle tip. The physical device tip and axis localization data 216 may be stored in data storage, for example, data storage 220. The physical device tip and axis localization data 216 may also be displayed on the display 218. In some embodiments, the physical device position information from system 200 can be displayed (e.g., on a display 218) to a physician as a virtual device during interventional procedures. Advantageously, the physical device position (e.g., tip position and axis orientation) estimated by system 200 can provide accurate feedback in real time during percutaneous interventions to guide or assist the physician and enable, for example, accurate manual device control or automatic remote control of device manipulation by robotic systems in the MRI system. In some embodiments, the physical device position data can also be used to update the MRI scan parameters of an MRI system (e.g., MRI system 100 shown in FIG. 1) to track and improve visualization of the physical device throughout different steps of the procedure. In addition, the estimated physical device position data 216 provided by system 200 can improve the physician's assessment of procedural accuracy during MRI-guided interventional procedure.

In some embodiments, the device feature or physical device tip and axis localization data 216 may be used for tracking the device feature or physical device position. For example, the device feature or physical device tracking results in X and Y directions in image coordinates may be calculated by subtracting the localized device feature or physical device position in an initial frame from the device feature or physical device localization results in each subsequent dynamic frame. In some embodiments, the image coordinate system can be transformed to the patient coordinate system based on the slice position.

In some embodiments, the two-stage device localization system 202 (including the device feature neural network 206, the images patch(es) generation module 208 and the physical device neural network 210), and the post-processing module 214 may be implemented on one or more processors (or processor devices) of a computer system such as, for example, any general-purpose computing system or device, such as a personal computer, workstation, cellular phone, smartphone, laptop, tablet, or the like. As such, the computer system may include any suitable hardware and components designed or capable of carrying out a variety of processing and control tasks, including steps for receiving image(s) of the subject 204, implementing the two-stage needle localization system 202 (including the device feature neural network 206, the image patch(es) generation module 208, and the physical device neural network 210), implementing post-processing module 214, providing the outputs 212 and tip and axis localization data 216 to a display 218 or storing the output(s) 212 and tip and localization 216 in data storage 220. For example, the computer system may include a programmable processor or combination of programmable processors, such as central processing units (CPUs), graphics processing units (GPUs), and the like. In some implementations, the one or more processor of the computer system may be configured to execute instructions stored in a non-transitory computer readable-media. In this regard, the computer system may be any device or system designed to integrate a variety of software, hardware, capabilities and functionalities. Alternatively, and by way of particular configurations and programming, the computer system may be a special-purpose system or device. For instance, such special-purpose system or device may include one or more dedicated processing units or modules that may be configured (e.g., hardwired, or pre-programmed) to carry out steps, in accordance with aspects of the present disclosure.

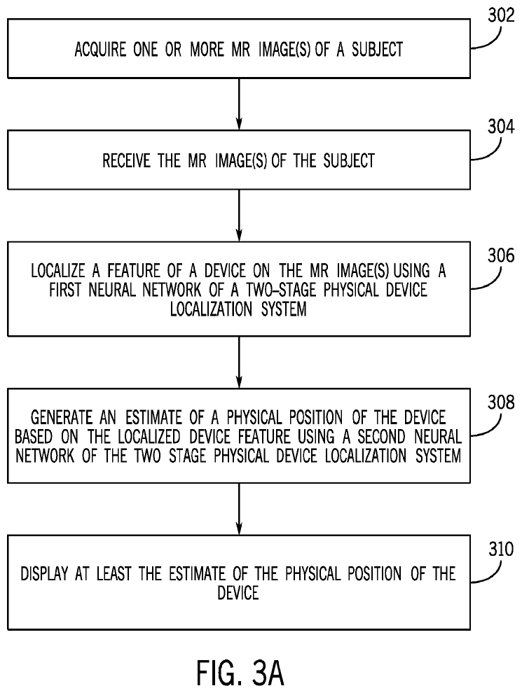

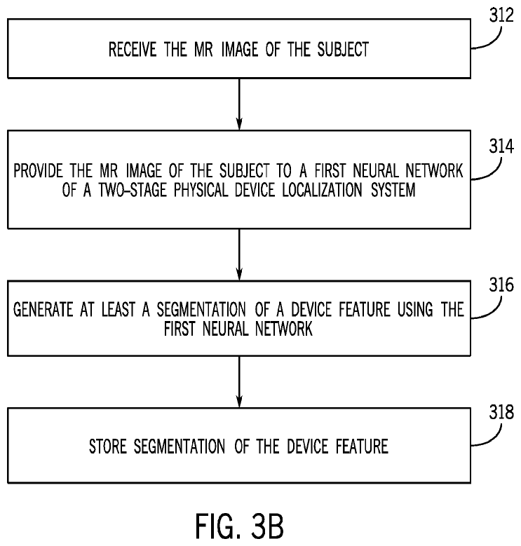

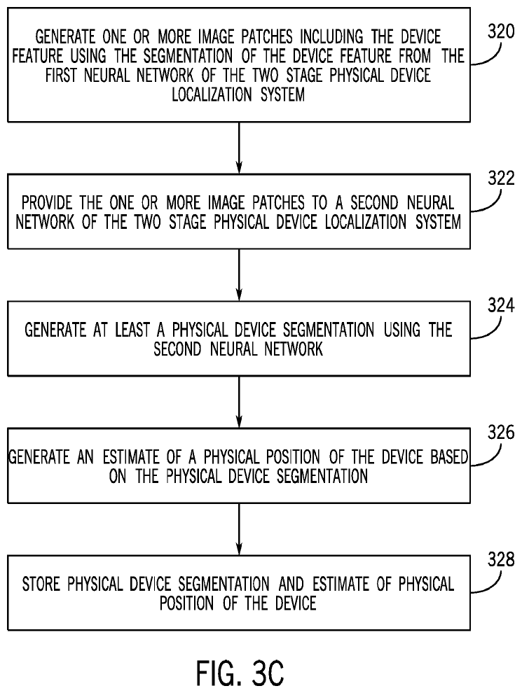

FIGS. 3A-3C illustrate a method for device localization and tracking in accordance with an embodiment. The process illustrated in FIGS. 3A-3C is described below as being carried out by the system 200 for system for device localization and tracking as illustrated in FIG. 2. Although the blocks of the process are illustrated in a particular order, in some embodiments, one or more blocks may be executed in a different order than illustrated in FIGS. 3A-3C, or may be bypassed.

In FIG. 3A, at block 302, one or more MR images 204 of a subject may be acquired using an MRI system (e.g., MRI system 100 shown in FIG. 1). In some embodiments, the MR image(s) 204 may be grayscale 2D MR image(s) that are stacked into three color channels. In some embodiments, the MR image(s) may be a stack of images encompassing the device (e.g., a needle) axis. In some embodiments, the MR image(s) 204 of the subject may be acquired in real-time from a subject (e.g., in vivo intra-procedural images) using an MRI system (e.g., MRI system 100 shown in FIG. 1), for example, for embodiments where the system 200 is utilized for device localization and tracking for MRI-guided interventions. For example, an MR image(s) 204 may be acquired from a subject using a pulse sequence performed on the MRI system and configured for interventional MRI. The interventional MRI sequence (and imaging parameters) used to acquire the MR image(s) 204 may be a known MRI interventional pulse sequence. In some embodiments, the MR image(s) 204 may be acquired using interactive real-time sequences configured to support a range of MRI-guided interventions. At block 304, the acquired MR image(s) may be received by a two-stage physical device localization system 202 from the MR system. The two-stage physical device localization system 202 may be configured to generate a physical device localization (e.g., estimating the physical device position) based on a device feature (e.g., a needle signal void feature). In some embodiments, the acquired MR image(s) 204 may be received in real-time from an MRI system (e.g., from data processing server 114 or disc storage 138 of MRI system 100 shown in FIG. 1), for example, the MRI system 100 may acquire MR data and reconstruct an MR image or images (e.g., using data processing server 114) using known reconstruction methods. In some embodiments, the MR image(s) 204 of the subject may be retrieved from data storage (or memory) of system 200, data storage of an imaging system (e.g., data storage of MRI system 100 shown in FIG. 1), or data storage of other computer systems (e.g., storage device 816 of computer system 100 shown in FIG. 10). For example, for embodiments where the system 200 is utilized for post-procedural analysis of device trajectory, the input MR image(s) 204 may be MR images acquired during an interventional procedure (i.e., procedural images) and stored in data storage.

At block 306, a feature of a needle (e.g., a signal void needle feature) may be localized on the MR image(s) 204 using a first neural network of a two-stage physical device localization system 202 as shown in FIG. 3B. In FIG. 3B, at block 312, the acquired MR image(s) 204 may be provided to a first stage of the two-stage physical device localization system 202 that may be implemented as a device feature neural network (e.g., a CNN such as a Mask R-CNN) 206. At block 314, the device feature neural network 206 may be used to generate at least a segmentation of the device feature. For example, the device feature neural network may be configured to detect and segment the device feature (e.g., the needle signal void feature) on the input MR image(s) 204. Advantageously, the device feature neural network 206 may detect and segment the device feature on the input MR image(s) at the instance-level, for example, by implementing the device feature neural network using a Mask R-CNN. In some embodiments, the detection and segmentation at block 316 by the device feature neural network 206 can generate a plurality of outputs including a class score, a bounding box, and a predicted mask. For example, the bounding box may indicate the region of interest (ROI) corresponding to the device feature detection and the predicted mask (within the bounding box) may correspond to the device feature segmentation. At block 318, at least the segmentation of the device feature generated by the device feature neural network 206 at block 316 may be stored in data storage, for example, data storage 220 (e.g., data storage of MRI system 100 shown in FIG. 1 or device storage 918 of computer system 900 shown in FIG. 9).

Returning to FIG. 3A, an estimate of a physical position of the device my be generated based on the localized device feature using a second neural network (e.g., a CNN such as a Mask R-CNN) 210 of the two-stage physical device localization system 202 as shown in FIG. 3C. In FIG. 3C, at block 320, one or more image patches including the device feature may be generated (e.g., with an image patch generation module 208) using the segmentation of the device features from the device feature neural network 206. In some embodiments, the MR image(s) 204 may be cropped (e.g. using image patch generation module 208) to a patch centered on the device feature (e.g., as identified by the detection and segmentation of the device feature neural network 206). In some embodiments, the cropped image patch is a single-slice image patch. In some embodiments, the image patch includes three adjacent and parallel image patches.

At block 322, the one or more image patch (e.g., a single-slice image patch or three adjacent and parallel slices) may be provided as an input to the physical device neural network 210 of the second stage of the two-stage physical device localization system 202. In some embodiments, the physical device neural network 210 may be configured as a single-slice physical needle Mask R-CNN model that takes a single-slice image patch as input. In some embodiments, the physical device neural network 210 may be configured as a 3-slice physical needle Mask R-CNN model that takes the three adjacent and parallel image patches as input. At block 324, at least a physical device segmentation may be generated using, for example, the physical device neural network 210. In some embodiments, the physical device neural network 210 can generate a plurality of output(s) 212 including, for example, a class score, a bounding box, and a predicted mask. For example, the bounding box may indicate the ROI corresponding to the physical device detection and the predicted mask (within the bounding box) may correspond to the physical device segmentation.

At block 326, an estimate of a physical position of the device (e.g., a needle) may be generated (e.g., using post-processing module 214) based on the physical device segmentation generated at block 324. In some embodiments, the estimate of the physical position or location of the device may be automatically determined or extracted by, for example, a post-processing module 214. For example, in some embodiments, the post-processing module 210 may be configured to calculate the physical device tip and axis orientation using the bounding boxe(es) of the physical device neural network outputs 212. For example, in some other embodiments, the post-processing module 214 may be configured to perform an orthogonal distance regression to localize the physical device tip and axis orientation from the segmentation mask(s) of the physical device neural network 210. At block 328, the physical device segmentation (e.g., an output 212 of the physical device neural network 210) may be stored in data storage, for example, data storage 220 (e.g., data storage of MRI system 100 shown in FIG. 1 or device storage 918 of computer system 900 shown in FIG. 9). Returning to FIG. 3A, at block 310, at least the estimate of the physical position of the device may be displayed, for example, on a display 218.

As mentioned above, in some embodiments, each of the two-stages 206, 210 of the two-stage physical device localization system 202 may advantageously be implemented using a Mask R-CNN. A Mask R-CNN architecture is configured to automatically integrate region of interest localization within the network which can direct the strong classification power of CNNs to the proposed regions instead of each pixel and enable the formation of an end-to-end learning framework for improved segmentation of, for example, the device feature or the physical device. The Mask R-CNN architecture also includes an additional mask branch to precisely determine the region containing the object feature while improving the robustness of the object feature segmentation task. While the following description of FIGS. 4-6B are discussed in terms of a Mask R-CNN, it should be understood that the device feature neural network 206 and the physical device neural network 210 may be implemented sing other types neural networks including other types of convolutional neural networks.

FIG. 4 is a block diagram of an example device feature mask region-based convolutional neural network (Mask R-CNN) 206 for the system for device localization and tracking of FIG. 2 in accordance with an embodiment. In FIG. 4, the device feature Mask R-CNN includes, a backbone architecture 430, a head architecture 432 and a region proposal network (RPN) 434. In the example shown in FIG. 4, in the backbone architecture 430, multi-scale features may be extracted. In some embodiments, the backbone architecture can include a Residual Network (ResNet) and a Feature Pyramid Network (FPN) that may be used to extract features from the entire input MR image(s) 204. The feature maps generated by the backbone architecture 430 may be shared with the region proposal network (RPN) 434 to generate an initial Region of Interest (ROI), indicated by a bounding box. In some embodiments, the RPN 434 may be initialized with 64 anchors for candidate region creation. The ROI and its corresponding feature map can be sent to the head architecture 432 for, for example, object classification, bounding box regression, and segmentation. In some embodiments, the head architecture 432 may include a ROIAlign layer which may be used to process the proposed ROI and corresponding feature map. The fixed-size ROI feature map may be exported for ROI refinement to obtain the outputs 436 including the final class score 438, ROI bounding box location 440, and predicted mask 442. Based on the bounding box location, the mask 442 can be transformed back into the original image size. In an embodiment, the device feature Mask R-CNN 206 can form an end-to-end supervised learning scheme for instance segmentation, which has advantages in detecting and segmenting small objects such as the device feature (e.g., needle feature) in a full field-of-view MR image. Compared with pixel-based segmentation, the disclosed device feature Mask R-CNN 206 requires no ensemble step to remove the residual errors from the output.

In some embodiments, other dark features in the anatomical background of the input MR image(s) 204 can mimic device (e.g., a needle) features and may be detected by the device feature Mask R-CNN 206, albeit with a lower class score. In some embodiments, to remove these false positive detection instances, a class score threshold (e.g., 0.99) may be set. In embodiments where only one device is being used during a procedure, if multiple positive ROIs are still detected after applying the threshold, the ROI with highest class score may be considered to be positive detection of the device. In some embodiments, if multiple positive device detection results from the device feature Mask R-CNN 206 are reported for a time frame, only the boundary box closest to the positively detected device feature in the previous time frame may be designated as the positive device detection result.

As discussed above, the device feature Mask R-CNN can be trained and configured to detect and segment the device feature (e.g., a needle signal void feature) on the input MR image(s) 204 (e.g., intra-procedural or real-time MR images). In some embodiments, the device feature Mask R-CNN 206 may be trained using a training dataset which may be generated by, for example, manually (e.g., by a radiologist) segmenting the device feature (e.g., a needle signal void feature) on MR images (e.g., in vivo MR images from, for example, interventional procedures). In addition, the MR image of the training dataset may also be annotated for the device feature tip location and axis orientation. In some embodiments, the device feature (e.g., a needle signal void feature) on 2D MR images may be defined as the only non-background class. In some embodiments, all images in the training dataset may undergo image augmentation such as, for example, by random rotation (0°-360°), flipping, translation, and rescaling to mitigate overfitting. In some embodiments, losses from the head architecture 432 and residual proposal network (RPN) 434 may be equally weighted. In some embodiments, for the training dataset, it may be assumed that the device has a rigid linear profile. In some embodiments, the device feature Mask R-CNN 206 may be pretrained using, for example, the common object in context (COCO) and phantom datasets to improve convergence during training for a training dataset with in vivo images. In some embodiments, the trained device feature Mask R-CNN 206 may achieve pixel-level tracking accuracy in real-time.

FIG. 5 is a block diagram of an example physical device Mask R-CNN for the system for device localization and tracking of FIG. 2 in accordance with an embodiment. In FIG. 5, the physical device Mask R-CNN 210 includes, a backbone architecture 552, a head architecture 554, and a region proposal network (RPN) 556. In the example shown in FIG. 5, in the backbone architecture 552, multi-scale features may be extracted. In some embodiments, the backbone architecture 552 can include a Residual Network (ResNet) and a Feature Pyramid Network (FPN) that may be used to extract features from the entire input image patch(es) 550 (e.g., generated by the image patch generation module 208 shown in FIG. 2). The feature maps generated by the backbone architecture 552 may be shared with the RPN 556 to generate an initial ROI, indicated by a bounding box. In some embodiments, the region proposal network (RPN) 556 may be initialized with 64 anchors for candidate region creation. The ROI and its corresponding feature map can be sent to the head architecture 554 for, for example, object classification, bounding box regression, and segmentation. In some embodiments, the head architect 554 may include a ROIAlign layer which may be used to process the proposed ROI and corresponding feature map. The fixed-size ROI feature map may be exported for ROI refinement to obtain the outputs 558 including the final class score 560, ROI bounding box location 562, and predicted mask 564. Based on the bounding box location 562, the mask 564 can be transformed back into the original image size. In an embodiment, the physical device Mask R-CNN 210 can form an end-to-end supervised learning scheme for instance segmentation, which has advantages in detecting and segmenting small objects such as the physical device in an MR image patch 550. Compared with pixel-based segmentation, the disclosed physical device Mask R-CNN 210 requires no ensemble step to remove the residual errors from the output.

As discussed above with respect to FIG. 2, the physical device neural network 210 may be trained and configured as a single-slice physical device Mask R-CNN that localizes the in-plane two-dimensional (2D) physical device tip and axis based on a single-slice image patch 550 or may be trained and configured as a 3-slice physical device Mask R-CNN model that localizes a three-dimensional (3D) physical device position (in-plane and through plane) based on three adjacent and parallel image patches 550. Training the physical device Mask R-CNN may require a substantial amount of data with reliable reference needle position information. In some embodiments, the physical device Mask R-CNN 210 (e.g., both the single-slice model and the 3-slice model) may be trained using a set of physics-based simulation images that can include realistic device-induced susceptibility features (e.g., needle signal void features). Accordingly, MRI physics-based simulations of the device susceptibility effects may be performed to generate a large set of training images that accurately depict the device feature with respect to actual intra-procedure MRI. In some embodiments, the disclosed training scheme may be applied to different interventional MRI sequences and imaging parameters by adjusting the simulation steps or by adjusting the simulation steps or by including the desired MRI parameters as inputs to the framework. In some embodiments, the set of MR training images may be simulated using known MR image simulation methods,

In one example, a Fourier-based off-resonance artifact simulation in the steady state (FORECAST) method may be implemented to simulate the training MR images (e.g., simulated single-slice MR images). For example, in some embodiments, the FORECAST method may be used to calculate the susceptibility effects in steady-state gradient echo (GRE) MRI. In this example, the field inhomogeneity or field shift ΔB₀(x, y, z) may be calculated as a function of different device orientations and device materials with different magnetic resonance properties using a first order perturbation approach to Maxwell's equations, combined with the Fourier transformation technique. In one example where the device is a needle, in the original FORECAST method, a thin slice with the desired FOV and slice thickness was modeled in 3D space, with the third dimension of ΔB₀ set to be parallel to B₀, which does not capture the tilting angle of the needle. To simulate the needle with a tilting angle, which is a more realistic scenario in interventional procedures, an expanded 3D model can be created. Specifically, in some embodiments, ΔB₀(x, y, z) may be calculated and re-sliced to an oblique volume parallel to the needle with certain excitation slice or slab thickness. A linear interpolation step may be performed to assign the ΔB₀ to each pixel of the model with the original pixel dimensions. In addition, a non-uniform fast Fourier transform (NUFFT) may be applied for a golden angle (GA) ordered radial sampling trajectory during the simulations. The overall k-space signal model of the needle susceptibility-induced effects on the discrete isochromatic grid with proton density ρ′(a, y, x) may be given by:

$$s ( {k}_{x} , {k}_{y} , {k}_{z} ) = {\sum }_{x} {\sum }_{y} {\sum }_{z} {ρ}^{′} ( x , y . z ) {e}^{- i 2 πγΔ {B}_{0} ( x , y , z ) {t}^{′} - {k}_{z} {t}^{′}} \times {e}^{- \frac{{t}^{′}}{{T}_{2}}} {e}^{- 2 π ( {k}_{x} x + {k}_{y} y )} \qquad Eq . 3$$

where γ is the gyromagnetic ratio and t′ is the time after radiofrequency (RF) excitation. In this example, the T₂* of the signal was decomposed into T₂ (e.g., 50 ms for muscle) and the field inhomogeneity caused by the needle susceptibility effects. Finally, an inverse NUFFT may be applied to the simulated k-space data to reconstruct the image, which contains the needle signal void feature.

For these simulations, in some embodiments, it may be assumed that the needle material is stiff enough and there is not deflection close to the tip. Therefore, the needle may be modeled as a cylindrical rod with diameter of, for example, 0.9 mm (20 gauge) at different rotation (θ) and tilting (α) angles in 3D space. In some embodiments, a range of θ (−30° to 30°) and α (0° to −90°) of the needle may be set according to actual reports of needle placement in percutaneous interventions.

In some embodiments, to ensure that the simulations matched the conditions of the needle used in a procedure, experimental data from a phantom (e.g., a gel phantom) with different needle orientations and imaging parameters may be used to calibrate the susceptibility value of the needle material. For example, simulated MR images that contained the needle feature with different rotation and tilting angles may be compared with the MR images from MRI-guided needle insertion experiments using a phantom. In some embodiments, the image-based needle susceptibility calibration method can compare the discrepancies between the physical needle and needle feature from experimental MRI data with the physics-based simulations in different situations. For example, in some embodiments, the Euclidean distance between the physical needle tip and the needle feature tip (dxy in mm) may be calculated for simulated data and phantom experimental data. The susceptibility value that minimized the average dxy may then be identified (e.g., a needle susceptibility value of 190 ppm corresponding to titanium) and used in subsequent simulations to generate the training data. This calibration step can improve the understanding of the needle feature characteristics under specific conditions and on specific types of MR images. The disclosed calibration techniques showed that the discrepancies between needle feature and physical needle varied with different needle orientations and imaging parameters. Proper selection of the needle susceptibility may ensure the fidelity of the simulated images for training.

As mentioned above, in some embodiments, the single-slice physical device Mask R CNN 210 model can take a single-slice image patch centered on the device feature and surrounding tissue as input, assuming the imaging plane is already closely aligned with the physical device. In some embodiments, using the single-slice physical device Mask R-CNN model in the two-stage physical device localization system 202 (shown in FIG. 2) can achieve sub-millimeter physical device localization accuracy on single-slice images aligned with the device (e.g., a needle). The two-stage physical device localization system 202 with single-slice physical device Mask R-CNN model can accurately and rapidly estimate the in-plane physical device position using single-slice MRI.

In some embodiments, ground truth labels for training of the single-slice physical device Mask R-CNN 210 may be structured as a 2D boundary box with corners that defined the physical device tip location and axis orientation. In some embodiments, the simulated single-slice training data for the single-slice physical device Mask R-CNN may be augmented using, for example, rescaling, translation, and adding Gaussian noise. With data augmentation, the simulated images can form a sufficient dataset to train the single-slice physical device Mask R-CNN while avoiding the need for expensive MRI experiments and manual annotation. In some embodiments, the training dataset for the single-slice physical device Mask R-CNN 210 may consist of simulated images with the same size as the expected input patches and the device feature tip in the center of the patch.

Interventional MR images acquired during actual procedural guidance can have more complex backgrounds compared with simulated images. In addition, certain types of tissue with off-resonance effects (e.g., fat) may also generate signal voids (e.g., fat-water signal cancellation) that occlude the device feature. These effects might degrade the accuracy of the two-stage physical device localization system 202 with the single-slice physical device Mask R-CNN 210 as the second stage. Therefore, after training with simulated data as previously described, in some embodiments fine-tuning of the single-slice physical device Mask R-CNN may be performed by using an additional training dataset with enriched variations of the background. In some embodiments, the additional (i.e. fine-tuning) dataset may be generated by acquiring MR images of ex vivo tissue in different slices without a device. Ex vivo tissue phantom MR images have realistic noise characteristics and also tissue features in the background, which resemble features expected on in vivo interventional MRI. Patches may then be randomly cropped from these ex vivo tissue images and superimposed with the simulated device images, followed by similar data augmentation as described above to increase the size of the fine-tuning dataset. Accordingly, the simulation images can be fused with the MR images for specific in vivo applications to form a fine-tuning dataset to further improve the performance of the single-slice physical device Mask R-CNN model.

As mentioned above, in some embodiments, the 3-slice physical device Mask R CNN 210 model can take three image patches 550, including device features from three adjacent parallel slices, in which the imaging plane orientation could be misaligned with the physical device axis. The three patches 550 may be stacked into three color channels of the network 202 input. In some embodiments, the bounding box output (e.g., bounding box location 562 shown in FIG. 5) may be increased from 4 to 6 dimensions to capture the 3D coordinates of the physical device's two ends (tip and entry point). In some embodiments, using the 3-slice physical device Mask R-CNN model in the two-stage physical device localization system 202 (shown in FIG. 2) can achieve physical device localization in real time to support, for example, MRI-guided interventions. The two-stage physical device localization system 202 with 3-slice physical device Mask R-CNN model can reduce the physical device axis orientation difference in the through-plane direction compared to the single-slice model. In some embodiments, the processing time of the two-stage physical device localization system 202 with 3-slice physical device Mask R-CNN model can be about 200 ms per set of 3-parallel-slice images, which is suitable for real-time interventional MRI applications. The 3-slice model can further reduce the through-plane needle localization error due to misaligned imaging plane with physical device (e.g., a needle) and rapidly estimated the overall 3D physical device position.

The 3-slice physical device Mask R-CNN may trained using physics-based simulated data of 3 adjacent parallel slices of MRI. In some embodiments, the simulated 3-slice training data for the 3-slice physical device Mask R-CNN may be augmented using, for example, rescaling, translation, and adding Gaussian noise. Ground truth labels for training may be structured as a 3D bounding box with corners that defined the physical device tip location and axis orientation. For simulating the training data using the method discussed above, in some embodiments, the thickness of the slab in the simulation model can be expanded from 5 mm to 15 mm to emulate three parallel imaging slices without any gap. In addition, misalignment between the device model and 3D acquisition slab may be characterized in the simulations by two additional geometric parameters: device-to-slice orientation difference (−) and pivot point (h) within the imaging slab. In some embodiments, the 3-slice physical device Mask R-CNN may be trained using training data including a combination of simulated data with ex vivo or inv vivo MR images.

In some embodiments, the single-slice and 3-slice physical device Mask R-CNN models described herein may be used in concert to support different steps in an MRI-guided interventional workflow. For example, during procedural setup and adjustment, a two-stage physical device localization system 202 with the 3-slice physical device Mask R-CNN model can be used to rapidly localize the physical device (e.g., a needle) position in 3D space and inform alignment of the MRI scan plane(s) with the physical device, using standard manual adjustments or new automated methods. Once the imaging plane is aligned with the physical device, the two-stage physical device localization system 2020 with the single-slice physical device Mask R-CNN model can be applied to dynamically localize, track, and confirm the physical device position with sub-millimeter accuracy. In some embodiments, this strategy for closed-loop confirmation of the physical device position can improve physicians' confidence in assessing and ensuring procedural accuracy.

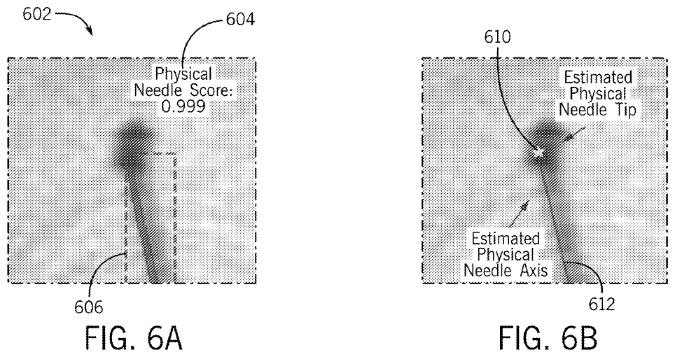

FIG. 6A shows an example physical needle detection and segmentation output for a physical needle Mask R-CNN in accordance with an embodiment and FIG. 6B shows an example physical needle localization output for a system for needle localization based on the physical needle detection and segmentation output of FIG. 6A in accordance with an embodiment. In FIG. 6A, the example physical needle detection and segmentation output 602 (e.g., output 212 shown in FIG. 2 or output 558 shown in FIG. 5) includes a class score 604 and a bounding box 606 overlaid on a region of interest of an MR image including the physical needle. In FIG. 6B, the example physical needle localization output 608 (e.g., physical needle tip and axis localization) shows an estimate of the physical needle tip 610 and an estimate of the physical needle axis 612.

Misalignment between the MRI scan plane and needle trajectory may degrade visualization and localization of the device. This may prolong procedure time and increase errors in MRI-guided interventions. Both a physician's visual perception of the device (e.g., a needle) and automatic device localization may be inadequate when the device feature is incomplete or missing due to misalignment between the MRI scan plane and underlying device position. In some embodiments, the disclosed system 200 (shown in FIG. 2) for device localization and tracking may be used in a system and method for automatically aligning an MRI scan plane or scan planes with a deice (e.g., a needle), which can further improve the localization performance of system 200 with enhanced device visualization. For example, in some embodiments, the disclosed technique for automatic scan plane alignment may enable new intra-procedural workflows that can automatically realign the MRI scan plane(s) with the device(s) throughout different steps of the procedure. Advantageously, the disclosed system and method do not require additional hardware to monitor the device for scan plane alignment.

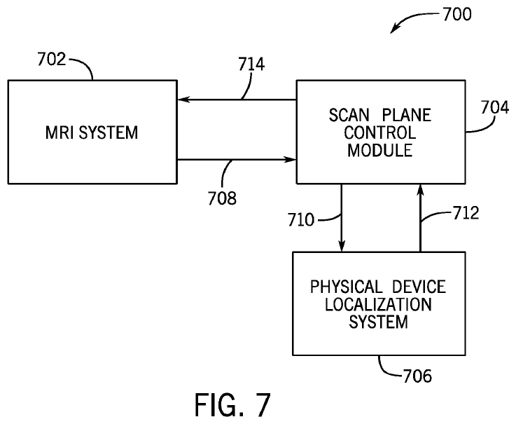

FIG. 7 is a block diagram of a system for automatic MRI scan plane alignment in accordance with an embodiment. The system 700 can include an MRI system (e.g., MRI system 100 shown in FIG. 1), a scan plane control (SPC) module 704 and a physical device localization system 706 (e.g., system 200 shown in FIG. 2). The SPC module 807 is coupled to and in communication with the MRI system 702 and the physical device localization system 706. In some embodiments, the SPC module 704 is configured to use device localization results from the physical device localization system 706 to automatically align the MRI scan plane(s) with the needle. The SPC module 704 may receive one or more MR image(s) 708 from the MRI system 702, acquired in real-time from an MR scan, for example during an interventional procedure. In some embodiments, the MR image(s) may have a moderate frame rate. If the MR system will be performing additional scans or protocols (i.e., the procedure is not complete), the SPC module 704 can provide the MR image(s) 710 to the physical device localization system 706. The physical device visualization system 706 can then be used to generate a physical device localization data or results (e.g., a segmentation, a physical device position (tip, axis)), for example, as described above with respect to FIGS. 2 and 3. The physical device localization data or results 712 may then be provided from the physical device localization system 706 to the SPC module 704. The SPC module 704 may be configured to determine an updated MRI scan plane based on the physical device localization data and results so that the scan plane is aligned with the device. In some embodiments, the SPC module determines and sets a new scan plane orientation and position, updates the scan parameters and provides the updated scan plane and scan parameters to the MRI system 702 which can then perform the next scan with the updated parameters.

In some embodiments, the scan plan control (SPC) module 704 and the physical device localization system 706 may be implemented on one or more processors (or processor devices) of a computer system such as, for example, any general-purpose computing system or device, such as a personal computer, workstation, cellular phone, smartphone, laptop, tablet, or the like. As such, the computer system may include any suitable hardware and components designed or capable of carrying out a variety of processing and control tasks, including steps for receiving image(s) of the subject, implementing the SPC module 704 and the physical device localization system 706. For example, the computer system may include a programmable processor or combination of programmable processors, such as central processing units (CPUs), graphics processing units (GPUs), and the like. In some implementations, the one or more processor of the computer system may be configured to execute instructions stored in a non-transitory computer readable-media. In this regard, the computer system may be any device or system designed to integrate a variety of software, hardware, capabilities and functionalities. Alternatively, and by way of particular configurations and programming, the computer system may be a special-purpose system or device. For instance, such special-purpose system or device may include one or more dedicated processing units or modules that may be configured (e.g., hardwired, or pre-programmed) to carry out steps, in accordance with aspects of the present disclosure

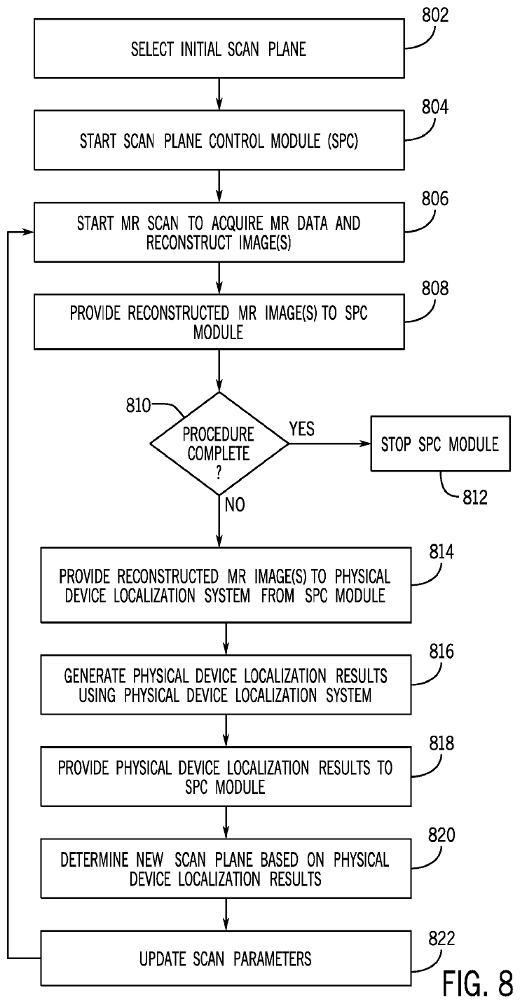

FIG. 8 illustrates a method for automatic MRI scan plane alignment in accordance with an embodiment. The process illustrated in FIG. 8 is described below as being carried out by the system 800 for system for automatic scan plane alignment as illustrated in FIG. 7. Although the blocks of the process are illustrated in a particular order, in some embodiments, one or more blocks may be executed in a different order than illustrated in FIG. 8, or may be bypassed

At block 802, an initial scan plane is selected, for example, based on a localizer scan. In some embodiments, the localizer scan can be a 3-plane localizer scan and the initial plane may be selected manually based on the device feature at the entry point on the 3-plane localizer images. At block 804 the scan plan control (SPC) module 704 is started and at block 806, the MR scan is started to acquire MR data and reconstruct MR image(s) using an MRI system 702 (e.g., MRI system 100 shown in FIG. 1). At block 808, the reconstructed MR image(s) are provided to the SPC module 704. At block 810, if the procedure is complete, the SPC module 704 can be stopped at block 812. If the procedure is not complete at block 810, the reconstructed MR image(s) may then be provided to a physical device localization system (e.g., system 200 shown in FIG. 2). At block 815, the physical device localization system 706 may generate physical device localization results (e.g., a segmentation, a physical device position (tip, axis)), for example, as described above with respect to FIGS. 2 and 3. At block 818, the physical device localization system 706 can provide the physical device (e.g., a needle) localization results to the SPC module 704. At block 820, an updated scan plane is determined by the SPC module 704 based on the physical device localization data and results so that the scan plane is aligned with the device. In some embodiments, the SPC module determines and sets anew scan plane orientation and position. At block 822, the SPC module updates the scan parameters and provides the updated scan plane and scan parameters to the MRI system 702 which can then perform the next scan with the updated parameters at block 806.

FIG. 9 is a block diagram of an example computer system in accordance with an embodiment. Computer system 900 may be used to implement the systems and methods described herein. In some embodiments, the computer system 900 may be a workstation, a notebook computer, a tablet device, a mobile device, a multimedia device, a network server, a mainframe, one or more controllers, one or more microcontrollers, or any other general-purpose or application-specific computing device. The computer system 900 may operate autonomously or semi-autonomously, or may read executable software instructions from the memory or storage device 816 or a computer-readable medium (e.g., a hard drive, a CD-ROM, flash memory), or may receive instructions via the input device 920 from a user, or any other source logically connected to a computer or device, such as another networked computer or server. Thus, in some embodiments, the computer system 900 can also include any suitable device for reading computer-readable storage media.

Data, such as data acquired with an imaging system (e.g., an MRI system, etc.) may be provided to the computer system 900 from a data storage device 916 or from the imaging system in real-time, and these data are received in a processing unit 902. In some embodiment, the processing unit 902 includes one or more processors. For example, the processing unit 902 may include one or more of a digital signal processor (DSP) 904, a microprocessor unit (MPU) 906, and a graphics processing unit (GPU) 908. The processing unit 902 also includes a data acquisition unit 910 that is configured to electronically receive data to be processed. The DSP 904, MPU 906, GPU 908, and data acquisition unit 910 are all coupled to a communication bus 912. The communication bus 912 may be, for example, a group of wires, or a hardware used for switching data between the peripherals or between any component in the processing unit 902.

The processing unit 902 may also include a communication port 914 in electronic communication with other devices, which may include a storage device 916, a display 918, and one or more input devices 920. Examples of an input device 920 include, but are not limited to, a keyboard, a mouse, and a touch screen through which a user can provide an input. The storage device 916 may be configured to store data, which may include data such as, for example, acquired data, acquired images, and device segmentation and localization data and images, whether these data are provided to, or processed by, the processing unit 902. The display 918 may be used to display images and other information, such as magnetic resonance images, patient health data, and so on.

The processing unit 902 can also be in electronic communication with a network 922 to transmit and receive data and other information. The communication port 914 can also be coupled to the processing unit 902 through a switched central resource, for example the communication bus 912. The processing unit can also include temporary storage 924 and a display controller 926. The temporary storage 924 is configured to store temporary information. For example, the temporary storage 924 can be a random access memory.

Computer-executable instructions for the systems and methods described herein may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

The present invention has been described in terms of one or more preferred embodiments, and it should be appreciated that many equivalents, alternatives, variations, and modifications, aside from those expressly stated, are possible and within the scope of the invention.

1. A system for device localization and tracking for magnetic resonance (MR) image-guided interventional procedures, the system comprising:
an input configured to receive a set of MR images of a region of interest of a subject acquired using a magnetic resonance imaging (MRI) system, wherein the region of interest includes a device;
a physical device localization system comprising:
a first neural network coupled to the input and configured to detect and localize a feature of the device on the set of MR images; and
a second neural network coupled to the first neural network and configured to generate an estimate of a physical position of the device based on the localized device feature; and
a display coupled to the second neural network and configured to display the estimate of the physical position of the device.

2. The system according to claim 1, further comprising a post-processing module coupled to the second neural network and configured to localize a tip and axis orientation of the device.

3. The system according to claim 1, further comprising a scan plane control module coupled to the physical device localization system and configured to determine an update of a scan plane of the MRI system to align the MRI system scan plane to the estimate of the physical position of the device.

4. The system according to claim 1, wherein the device feature is a device feature caused by device-induced susceptibility effects.

5. The system according to claim 4, wherein the device feature is a signal void.

6. The system according to claim 1, wherein the estimate of the physical device position is displayed on at least one MR image of the region of interest of the subject.

7. The system according to claim 1, wherein the localized device feature is provided to the second neural network as an image patch containing the device feature.

8. The system according to claim 1, wherein the device is a needle.

9. The system according to claim 1, wherein the first neural network is a convolutional neural network.

10. The system according to claim 9, wherein the first neural network is a mask region-based convolutional neural network (Mask R-CNN).

11. The system according to claim 1, wherein the second neural network is a convolutional neural network.

12. The system according to claim 9, wherein the second neural network is a mask region-based convolutional neural network (Mask R-CNN).

13. The system according to claim 12, wherein the second neural network is a single-slice Mask R-CNN configured to receive a single-slice image patch containing the device feature.

14. The system according to claim 12, wherein the second neural network is a 3-slice Mask R-CNN configured to receive three adjacent and parallel image patches, wherein at least one of the image patches contains the device feature.

15. The system according to claim 10, wherein the first neural network includes a backbone architecture and a head architecture.

16. The system according to claim 12, wherein the second neural network includes a backbone architecture and a head architecture.

17. The system according to claim 12, wherein the second neural network is trained using a plurality of simulated training images that include the device feature.

18. The system according to claim 1, wherein the estimate of the physical position of the device is generated in real-time.

19. A method for device localization and tracking for magnetic resonance (MR) image-guided interventional procedures, the method comprising:
acquiring, using a magnetic resonance imaging (MRI) system, a set of MR images of a region of interest of a subject, wherein the region of interest includes a device;
detecting and localizing a feature of the device on the set of MR images using a first neural network;
generating an estimate of a physical position of the device based on the localized device feature using a second neural network coupled to the first neural network; and
displaying, using a display, the estimate of the physical position of the device.

20. The method according to claim 19, further comprising localizing, using a post-processing module, a tip and axis orientation of the device.

21. The method according to claim 19, further comprising determining, using a scan plane control (SPC) module, an update of a scan plane of the MRI system to align the MRI system scan plane to the estimate of the physical position of the device.

22. The method according to claim 19, wherein displaying the estimate of the physical device position include displaying the estimate of the physical device position on at least one MR image of the region of interest of the subject.

23. The method according to claim 19, wherein the second neural network is a single-slice mask region-based convolutional neural network (Mask R-CNN) configured to receive a single slice image patch containing the device feature.

24. The method according to claim 19, wherein the second neural network is a 3-slice mask region-based convolutional neural network (Mask R-CNN) configured to receive three adjacent and parallel image patches, wherein at least one of the image patches contains the device feature.

25. The method according to claim 19, wherein the estimate of the physical position of the device is generated in real-time.

26. The method according to claim 19, wherein the device is a needle.
